(19) United States
(12) Patent Application Publication
(10) Pub. No.: US 2022/0062569 A1
(43) Pub. Date: Mar. 3, 2022

(54) ULTRASONIC DIFFUSER APPARATUS AND RELATED SYSTEM AND METHOD OF ADMINISTERING WATER-COMPATIBLE NANOEMULSIONS

(71) Applicants: Brandon Moreau, Lauderdale-by-the-Sea, FL (US); Jonathan Moreau, Miami, FL (US); Sheldon Pink, Boca Raton, FL (US); Luke Paget, Flagstaff, AZ (US); David W Kidd, IV, Strong, ME (US)

(72) Inventors: Brandon Moreau, Lauderdale-by-the-Sea, FL (US); Jonathan Moreau, Miami, FL (US); Sheldon Pink, Boca Raton, FL (US); Luke Paget, Flagstaff, AZ (US); David W Kidd, IV, Strong, ME (US)

(21) Appl. No.: 17/462,788

(22) Filed: Aug. 31, 2021

Related U.S. Application Data
(60) Provisional application No. 63/072,984, filed on Sep. 1, 2020.

(51) Int. Cl. A61M 15/00; A61K 31/05; A61K 9/51

(52) U.S. Cl. CPC A61M 15/0085; A61K 31/05; B82Y 5/00; A61M 2205/50; A61M 2205/0294; A61K 9/5192

(57) ABSTRACT

An ultrasonic diffuser apparatus uses ultrasonic waves to aerosolize nano-emulsified cannabinoids into airborne nanoparticles. The apparatus includes at least: a base housing to house electronic components, a CBD storage compartment communicative with the base housing, and a cover removably mated with the CBD storage compartment. The cover includes an interior cavity and an exhaust outlet. The aerosolized CBD oil solution collects within the interior cavity and is expelled through the exhaust outlet where it is absorbed through the skin, mucus membranes and lungs of persons in a substantially closed room.

### CROSS-REFERENCE TO RELATED APPLICATIONS

This application is a non-provisional of, and claims priority to, U.S. Application Ser. No. 63/072,984, filed on Sep. 1, 2020, titled “Ultrasonic Diffuser Apparatus and Related System and Method of Administering Water-Compatible Nanoemulsions;” which application is incorporated by reference in its entirety.

### FIELD OF THE INVENTION

The present invention generally relates to diffusers, and more specifically relates to an ultrasonic diffuser apparatus for passively administering cannabis oil and related system and method of use, thereof.

### BACKGROUND OF THE INVENTION

Currently, inhalation therapies rely on direct inhalation nebulizers that nebulize salbutamol, albuterol, and other medications for patients with lung conditions such as asthma. Nebulizers are an excellent medicine delivery medium but they run the risk of contamination through their mouthpiece and/or facemask.

Recently, it has been reported that cannabidiol (CBD), a compound derived from hemp plants, provides numerous health benefits. CBD and Tetrahydrocannabinol (THC) are both cannabinoids from the hemp plant. However, unlike THC, CBD is not psychoactive, propelling its growing use for medical treatment. Vape pens have become popular as a delivery medium for CBD; however the Centers for Disease Control (CDC) recently issued a warning concerning vape pens.

Ultrasonic diffusers are known in the art for those that wish to fill a room with scent particles. Ultrasonic diffusers are quieter and less expensive than nebulizers and are used frequently in aromatherapy applications. CBD oil particles, however, are heavier than those used for aromatherapy and are not well suited for airborne dispersal via ultrasonic diffusers since the CBD particles stay behind and only the water is diffused, resulting in no beneficial or therapeutic effects to the user.

There exists, therefore, a need for a safe and sanitary therapeutic treatment modality to overcome the above-stated shortcomings of the known art.

### SUMMARY OF THE INVENTION

Briefly, an embodiment of the present invention is directed to an ultrasonic diffuser apparatus adapted to use ultrasonic waves to aerosolize nano-emulsified cannabinoids into airborne nanoparticles. The ultrasonic diffuser apparatus includes, inter alia, a base housing configured to house electronic components including a power supply, a user interface, at least one piezoelectric disk, and a fan; a CBD storage compartment communicative with the base housing and configured to contain nano-emulsified CBD oil solution in sonic communication with the piezoelectric disk; and a cover removably mated with the CBD storage compartment. The cover includes an interior cavity and an exhaust outlet. The aerosolized CBD oil solution collects within the interior cavity and is expelled through the exhaust outlet where it is absorbed through the skin, mucus membranes and lungs of persons in a substantially closed room.

According to another embodiment, a method for the non-invasive, smoke-free, therapeutic treatment of ailments treatable by cannabinoids includes: providing the ultrasonic diffuser apparatus, as above; providing nano-emulsified CBD oil solution to deliver between 0.1 mg and 10 mg of CBD per square foot plus between 1 mg and 100 mg of CBD per person in a substantially closed room; and powering on the apparatus to impart frequencies into the nano-emulsified CBD oil solution such that the interior cavity of the cover is saturated with the nebulized solution and is exhausted through the exhaust outlet until a desired saturation level is achieved.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 is a schematic diagram of the base of the ultrasonic diffuser apparatus, according to an embodiment;

FIG. 2 is a schematic diagram of the device chamber, according to an embodiment;

FIG. 3 is a schematic diagram of the device storage component (basin), according to an embodiment;

FIG. 4 is a schematic diagram of the circuit for the ultrasonic diffuser apparatus, according to an embodiment;

FIG. 5 is an operational flow diagram of a method for treatment using an ultrasonic diffuser apparatus to disperse nano emulsified Cannabinoids such as CBD, CBC, CBN, THC and THC-a;

FIG. 6 is a product rendering of the ultrasonic diffuser apparatus, according to an embodiment;

FIG. 7 shows a diagram of the base with dimensions, according to an embodiment;

FIG. 8 shows a diagram of the cover with dimensions, according to an embodiment;

FIG. 9 shows a view of the front of the tank with dimensions, according to an embodiment;

FIG. 10 shows an orthogonal view of the tank with dimensions, according to an embodiment; and

FIG. 11 shows a top view of the tank with dimensions, according to an embodiment.

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention. Additionally, any dimensions shown on the various figures are for reference only and are provided to give context to the particular embodiment shown and not to limit the scope of the invention in any way, except as is claimed.

### DESCRIPTION OF THE INVENTION

The invention disclosed herein provides a non-invasive, therapeutic treatment apparatus, system, and method for the treatment of a multitude of ailments that cannabinoids and terpenes can help alleviate. Preferred embodiments of the invention include an ultrasonic nebulizing apparatus that acts on a solution of nano-emulsified Cannabinoids such as CBD CBC and THC. Terpenes are natural compounds that give the hemp plant its smell and flavor. They may be combined with the CBD oil to create flavors. Terpenes are also thought to enhance the beneficial effects of CBD in what is known as the “entourage effect.” Nano-emulsifying CBD oil helps to increase the amount of the CBD that actually gets into the bloodstream.

The nebulized nano-emulsified cannabinoids are expelled from the nebulizing apparatus, saturating a room with airborne nano-emulsified cannabinoid droplets that can be inhaled orally or nasally by an occupant of the room. In this way, the user has to simply walk in and inhale. There is no need for a mask, pen, inhaler, or other traditional delivery modality.

Those with ordinary skill in the art will appreciate and recognize that this apparatus, system, and method may employ any nano-emulsified oil (eucalyptus, etc. . . . ) and is not limited to CBD oil.

Furthermore, where other systems and methods as known in the art use apparatus that are 1-to-1, meaning one apparatus per one user, this invention can use one apparatus per room and, therefore, can be used by multiple people at the same time.

As used herein, the terms nebulize and diffuse (and their grammatical equivalents, such as, but not limited to, nebulizer and diffuser) are used interchangeably to mean to aerosolize a substance that is in a liquid state, including a solution and/or a suspension and/or an emulsion, into nanoparticles that are dispersed into the air.

The Ultrasonic Nebulizer

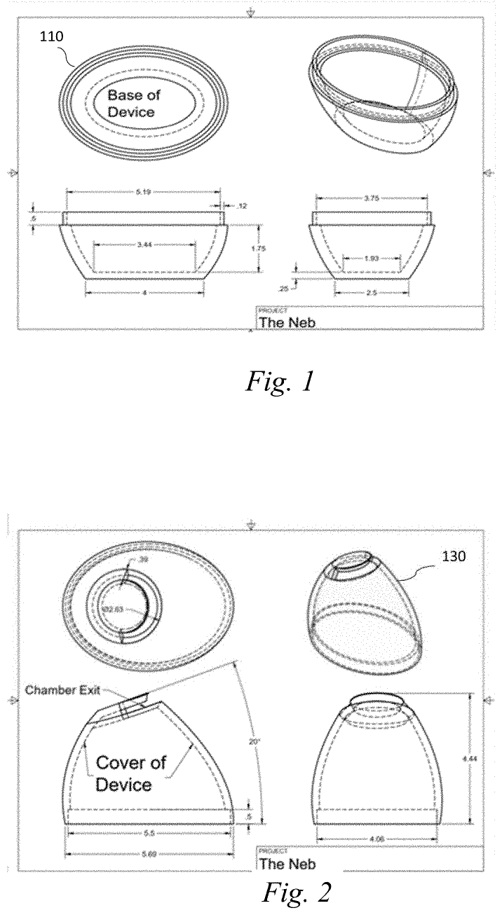

Referring now to FIGS. 1-4 and FIGS. 6-10, we describe an ultrasonic nebulizer that uses ultrasonic waves to aerosolize nano emulsified cannabinoids into nanoparticles that are dispersed into the air so that they are absorbed into the body of a person who breathes in, or is otherwise exposed to, the CBD/CBC/THC-laden air. The ultrasonic nebulizer has, at least, a base housing 110, electronic and electrical components configured to produce soundwaves in the ultrasonic frequencies 140, a CBD storage compartment 120, and a cover 130.

The base housing 110 is configured to house the electronics 140 that are used to create the ultrasonic sound waves. Additional electronics are also contained within the base housing 110, such as, but not limited to, a power supply/power regulator, a user interface (such as one or more buttons, status lights, and the like), and a fan.

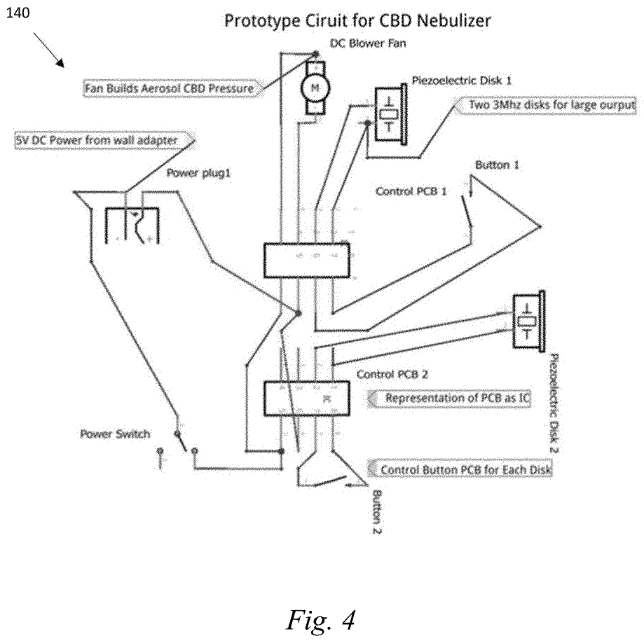

Referring to FIG. 4 in particular, by way of example and not limitation, a preferred embodiment of the invention comprises one or more piezoelectric disks in electrical communication with a power supply. The piezoelectric disks are to be chosen from those known in the art to produce frequencies in the 0.7 MHz to 2.7 MHz range. In this preferred embodiment, each piezoelectric disk is coupled with its own control button, or switch, such that each piezoelectric disk may be selectively turned on or off by a user. In this way, the user can select the amount, or strength, of CBD or other cannabinoids expelled into the surrounding air.

The apparatus also includes a power supply circuit configured to supply electrical power to the switches, piezoelectric disks, and other electrical and electronic components. In embodiments, the power supply may be configured to receive 5V DC power from an external adaptor, but the AC to DC conversion can also be accomplished within the apparatus.

Additionally, embodiments of the apparatus include a DC blower fan. The fan is powered by the power supply and is configured so as to propel the aerosolized particles from the apparatus into the surrounding air.

A CBD storage compartment 120 is communicative with the base housing 110. The CBD storage compartment 120 is configured to contain an amount of nano-emulsified CBD oil solution in sonic communication with the one or more piezoelectric disks.

A cover 130 is configured to be removably mated with the CBD storage compartment 120. The cover 130 is further configured with an exhaust outlet. The interior cavity of the cover 130 is configured so as to collect the aerosolized CBD oil solution and, as the pressure builds in the cover 130, the aerosolized solution is forced to escape, be expelled, through the exhaust opening. In embodiments, the cover 130 may be shaped so as to provide a directional expulsion of the aerosolized solution.

In one embodiment, nebulizing discs can be placed inside the basin 120. The Exhaust port for an aerosolized solution can be rotated around its current location or its size could be changed. Micro-controllers located inside could be switched with any microcontroller that can operate piezoelectric discs at the required frequency 0.7-2.7 Mhz for extended periods of time.

The device overcomes the above-stated shortcomings of the prior art by providing a non-invasive, sanitary, therapeutic treatment for a multitude of ailments that cannabinoids and terpenes can help alleviate. It is smoke-free and eliminates harmful heated plant matter and carcinogens that come with traditional inhalation delivery methods. This product is able to saturate a room with nano emulsified cannabinoid particles that will be passively absorbed by the lungs simply through breathing while inside the room. Multiple people can be treated at the same time. This allows the CBD nebulizer to be used not only as a traditional CBD treatment for various related health factors but also as a passive therapy for chronic asthmatics, epilepsy patients and others suffering from serious pulmonary illnesses.

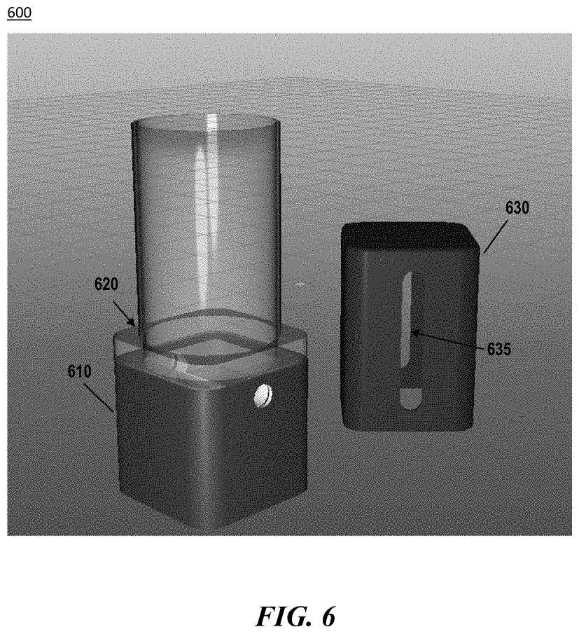

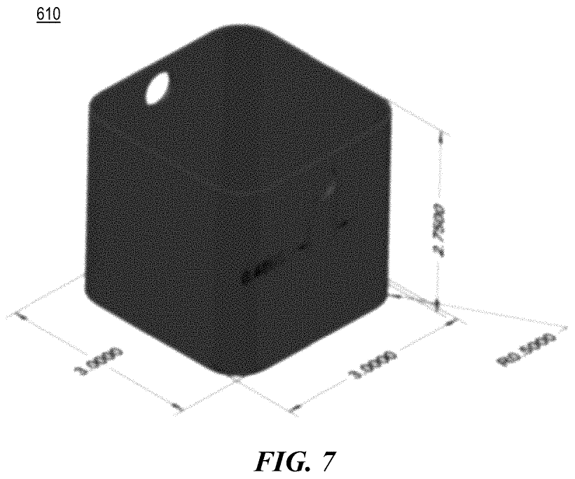

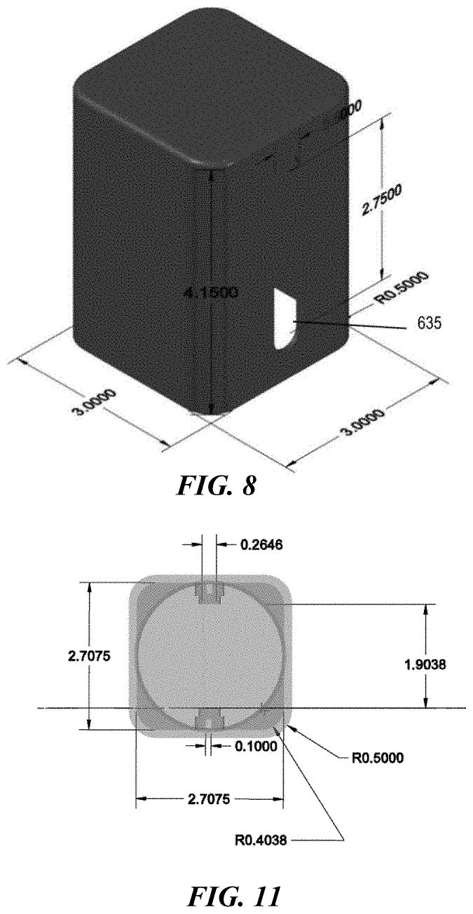

Referring now to FIGS. 6 through 11, we show several views of another embodiment of the ultrasonic nebulizer 600. FIG. 6 shows the three main components: the base housing 610 which houses the electronics 640 (not shown), the cover 630, and the CBD storage compartment 620. Non-limiting dimensions are provided merely for context. FIG. 7 shows the base housing 610 with suggested dimensions, according to one embodiment. The base 610 in this embodiment is substantially cube shaped, with a size of approximately three inches. FIG. 8 shows the cover 630 with an elongated opening along the side (the exhaust outlet 635). Note that the exhaust outlet 635 is shown as an elongated opening along the side of the cover 630 in this particular embodiment; however, the exhaust outlet 635 can also be configured as an opening on the top of the cover and it can be any other shape so long as the nano-particles can escape through the exhaust outlet 635. The dimensions of the cover 630 in this embodiment are approximately 3 inches wide by 3 inches long with a height of approximately 3½ inches.

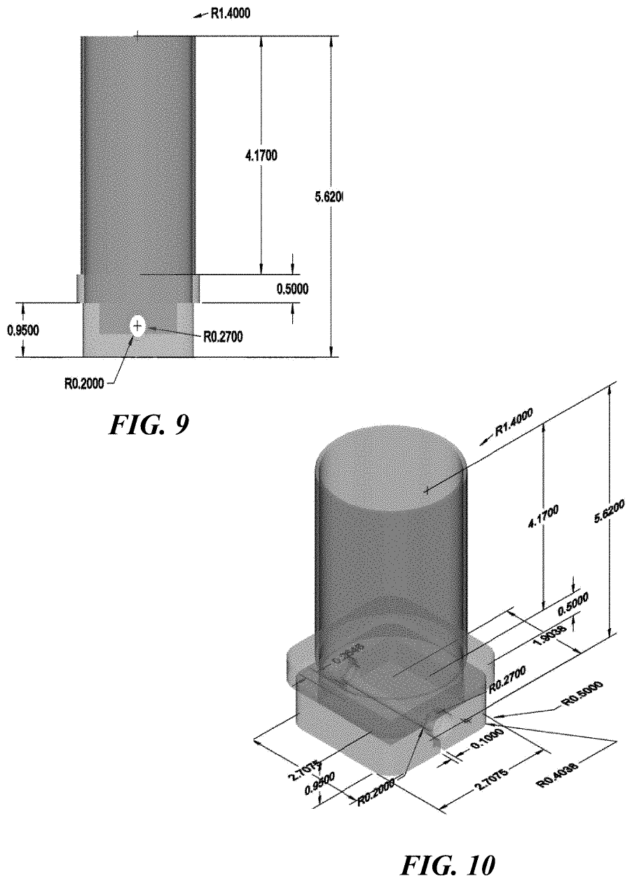

FIG. 9 shows the storage compartment 620 coupled with the base housing 610. FIG. 10 shows an orthogonal view of the storage compartment 620 and base housing 610. FIG. 11 shows a view of the top of the storage compartment 620.

System

A system for the delivery of nano emulsified cannabinoids includes a nebulizer apparatus, as disclosed above. The system further includes an effective amount of nano emulsified CBD, CBN, CBC, THC, etc. in a liquid solution and a room. For improved efficacy of delivery, the room should be substantially enclosed so as to limit the amount of aerosolized CBD that can escape the room.

Method

Cannabis/CBD has multiple delivery vectors/modalities:
oral (digestive): via edibles and gel capsules;
pulmonary: smoking, vaping, dabbing, nebulizer (inhaler);
transmucosal: sublingual, intranasal, ocular, rectal; and
transdermal: via creams, ointments, patches, topicals.

The typical “bioavailability” (the amount of cannabinoid content that can be absorbed into the bloodstream) of each method is typically small since cannabinoids are not water-soluble and, therefore, are not readily compatible with the predominantly water-based human physiology. Utilizing the apparatus and system disclosed in this invention, as opposed to those known in the prior art, provides multiple delivery vectors/modalities, thereby increasing the bio-availability and effectiveness of the apparatus and system to provide results via the treatment method.

Our unique solution of saturating a room with an aerosolized CBD solution and allowing a user or users to simply be present in the room and inhale has not been previously conceived. As discussed, above, the device works by using a high frequency to break down a nano emulsified cannabinoid solution into a fine aerosol (2-5 microns in size). Those particles fill a chamber and then get expelled from the chamber into the air as the chamber fills with the aerosolized solution. This will cause a room to be filled with aerosolized cannabinoids that can be metabolized by the pulmonary system, the skin, and the mucus membranes.

Dosing of the CBD solution may be customized for each user and/or treatment plan. For example, between 0.1 mg and 10 mg of CBD per square foot plus between 1 mg and 100 mg of CBD per person in one room. A preferred embodiment of the method provides about 1 mg of CBD per square foot plus about 40 mg of CBD per person in one room. Tests have been conducted utilizing 150 ml of solution with 80 mg of CBD for an 80 square foot room.

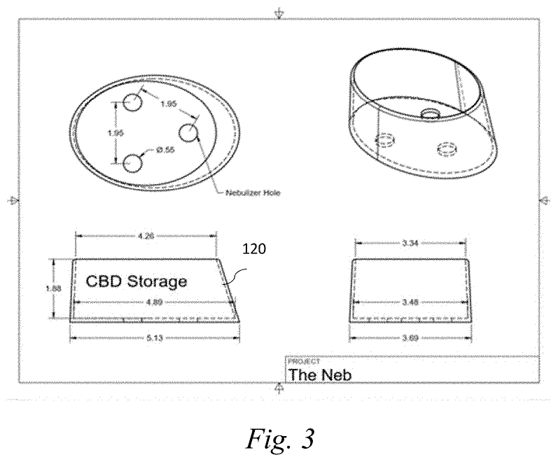

The CBD/Terpene solution is placed in a basin 120, as shown in FIG. 3. and is gradually dispersed into the air as an aerosol. Referring to FIG. 3, in one embodiment the device features three 1.7 MHz ultrasonic nebulizers which are placed at the bottom of the basin 120 where the solution is deposited.

When the device is activated, it powers the nebulizers and saturates the inside of the chamber 130 quickly. At this point a fan will be pushing the solution out through an exhaust port near the top of the device 130. This exhaust will then saturate the room to a desired level at which point the device will halt nebulization. Desired saturation level is achieved when a room is foggy, at this point the vapor is being absorbed through the skin, mucus membranes, and lungs.

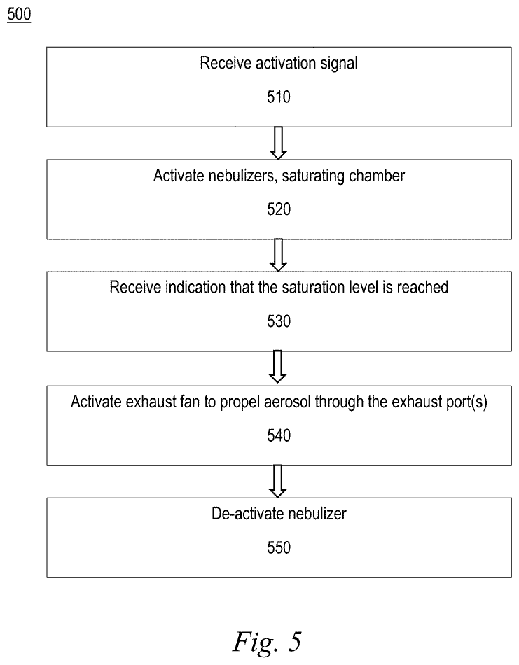

Referring now to FIG. 5 there is shown an operational flow diagram 500 of a method for dispersing nano emulsified cannabinoids according to an embodiment of the present invention. The method begins at step 510 when the electronic circuitry 140 receives a signal that the device has been turned on. Responsive to receiving the activation signal, in step 520, the nebulizers are activated, acting on the nano emulsified CBD/Terpene solution in the basin 120, generating aerosolized CBD in a fine mist, saturating the chamber 130. Once a saturation level is reached in step 530, the exhaust fan is activated in step 540, propelling the aerosolized CBD through the exhaust port and out into the surrounding air. In step 550, the nebulizers are de-activated when sensors within the apparatus detect that the apparatus is out of solution. Alternatively, the device may also contain a timer circuit to shut off the unit after a prescribed amount of time so long as there is enough solution in the device.

Multiple uses of the device are contemplated, within the spirit and scope of the invention. This product could be utilized as a medical treatment in hospitals. Spas and alternative health centers could use this alongside current treatments. In the hospital scenario, we would expect to have a larger output of nebulized solution for higher dosage treatments, and for multiple people or groups to be receiving treatment at the same time. Our home-based product would have more commodities with a simple user interface for easy use. Differing terpenes and additives will make each product line distinguishable from a recreational commodity to real medical treatment.

In light of the foregoing description, it should be recognized that embodiments in accordance with the present invention can be realized in numerous configurations contemplated to be within the scope and spirit of the claims. Additionally, the description above is intended by way of example only and is not intended to limit the present invention in any way, except as set forth in the claims.

Modifications and changes to the invention should be readily apparent to those having ordinary skill in the art, which modifications are intended to be within the spirit and scope of the invention as claimed. It is also understood that the foregoing description is illustrative of the present invention and should not be considered as limiting. Components from one embodiment can be combined with another embodiment and remain within the spirit and scope of the invention. Therefore, other embodiments of the present invention are possible without departing from the spirit and scope of the present invention.

1. An apparatus adapted to use ultrasonic waves to aerosolize nano-emulsified cannabinoids into airborne nanoparticles, the apparatus comprising:
a base housing configured to house a one or more electronic or electrical component including a power supply, a user interface, or a fan;
a one or more piezoelectric disk configured to produce frequencies in the 0.7 MHz to 2.7 MHz range, inclusive, where the one or more piezoelectric disk is disposed within the base housing;
a CBD storage compartment communicative with the base housing and configured to contain an amount of nano-emulsified CBD oil solution in sonic communication with the one or more piezoelectric disks; and
a cover configured to be removably mated with the CBD storage compartment, where the cover further includes an interior cavity and an exhaust outlet such that the interior cavity of the cover is configured so as to collect aerosolized CBD oil solution and, as the pressure builds within the cover, the aerosolized CBD solution is forced to be expelled through the exhaust outlet.

2. The apparatus of claim 1 further comprising a one or more microcontroller adapted to operate the one or more piezoelectric disk.

3. The apparatus of claim 1 wherein the exhaust outlet is adapted to produce a directional expulsion of the aerosolized CBD solution.

4. A method for non-invasive, smoke-free, therapeutic treatment of ailments so treatable by cannabinoids adapted for the simultaneous treatment of multiple people in a substantially closed room, the method comprising:
providing an apparatus adapted to use ultrasonic waves to aerosolize nano-emulsified cannabinoids into airborne nanoparticles, the apparatus comprising:
a base housing configured to house a one or more electronic or electrical component including a power supply, a user interface, or a fan;
a one or more piezoelectric disk configured to produce frequencies in the 0.7 MHz to 2.7 MHz range, inclusive, where the one or more piezoelectric disk is disposed within the base housing;
a CBD storage compartment communicative with the base housing and configured to contain an amount of nano-emulsified CBD oil solution in sonic communication with the one or more piezoelectric disks; and
a cover configured to be removably mated with the CBD storage compartment, where the cover further includes an interior cavity and an exhaust outlet such that the interior cavity of the cover is configured so as to collect aerosolized CBD oil solution and, as the pressure build in the cover, the aerosolized CBD oil solution is forced to be expelled through the exhaust outlet;
providing an amount of nano-emulsified CBD oil solution to deliver between 0.1 mg and 10 mg of CBD per square foot plus between 1 mg and 100 mg of CBD per person in the substantially closed room;
powering on the apparatus such that the one or more piezoelectric disk imparts frequencies into the amount of nano-emulsified CBD oil solution;
saturating the interior cavity of the cover with the nebulized solution;
exhausting of the nebulized solution through the exhaust outlet into the room until a desired saturation level is achieved; and
absorbing through the skin, mucus membranes and lungs of the multiple people in the substantially enclosed room;
whereby, the method provides for the non-invasive, smoke-free, therapeutic treatment of ailments so treatable by cannabinoids adapted for the simultaneous treatment of multiple people in a substantially closed room.

5. The method of claim 4 wherein the amount of nano-emulsified CBD oil solution contains one or more additives.
